(19) United States
(12) Patent Application Publication
(10) Pub. No.: US 2022/0272969 A1
(43) Pub. Date: Sep. 1, 2022

(54) ANTIMICROBIAL SUBSTRATE

(71) Applicant: Berkshire Corporation, Greensboro, NC (US)

(72) Inventors: TODD SHOWALTER, GREENSBORO, NC (US); SUSAN FAILE, GREENSBORO, NC (US); NATHAN HEATON, GREENSBORO, NC (US); WENYU ZHANG, GREENSBORO, NC (US)

(73) Assignee: Berkshire Corporation, Greensboro, NC (US)

(21) Appl. No.: 17/652,373

(22) Filed: Feb. 24, 2022

Related U.S. Application Data
(60) Provisional application No. 63/154,007, filed on Feb. 26, 2021.

(51) Int. Cl. A01N 25/34; A01N 25/10; A01N 43/90; A01P 1/00

(52) U.S. Cl. CPC A01N 25/34; A01N 25/10; A01N 43/90; A01P 1/00

(57) ABSTRACT

A portion of personal protective equipment treated with a topically applied finish containing a reactive oxygen species-generating dye (ROS) activated by electromagnetic radiation in the visible light region coated on the substrate with the ROS dye incorporated between 0.01-5.00% on weight of substrate to provide antimicrobial or self-cleaning benefits to the textile substrate.

This non-provisional patent application claims all benefits under 35 U.S.C. § 119(e) of pending U.S. provisional patent application Ser. No. 63/154,007 filed 26 Feb. 2021, entitled “ANTIMICROBIAL SUBSTRATE”, in the United States Patent and Trademark Office, which is incorporated by reference in its entirety herein.

### FIELD OF THE INVENTION

The invention herein pertains to one or more substrates imbued, embedded, or otherwise bestowed with certain antimicrobial properties, and particularly pertains to an article of personal protective equipment such as a textile facemask carrying a dye or resin activated by electromagnetic radiation in a predetermined volume and/or concentration sufficient to impart desired antimicrobial efficacy against a broad variety of micro-organisms, including bacteria, viruses, and/or fungi to the substrate.

### DESCRIPTION OF THE PRIOR ART AND OBJECTIVES OF THE INVENTION

Micro-organisms including (but not limited to) bacteria, viruses, parasites, and fungi can be found on nearly every surface in everyday life, including water, food, and the human body, as well as surfaces included to protect the foregoing such as masks, garments, and textiles. Micro-organisms may be odorless, tasteless, and/or invisible to the naked eye but they proliferate easily, may become resistant to certain treatments designed to kill them, and have the ability to grow rapidly. The rapid growth of micro-organism populations may be especially problematic in, or on textile(s). Textiles in the nature of clothing protect the human body against external factors. However, because textiles are typically unsterilized, they can possess a high amount of bacteria as sweat and other forms of fluids containing bacteria are transferred from the skin. Previous attempts to remedy this circumstance have met with limited success. For example, it is generally known to incorporate a wide range of antimicrobial finishes and/or formulas to textiles using binding agents to bestow upon the textile antimicrobial properties. However, the chemical means necessary to achieve attachment are expensive, inconsistent, and not industrially relevant. Similar finishes in the prior art have attached various agents to textiles of different composition to provide antimicrobial or self-cleaning benefits. These include numerous agents based on quaternary ammonium compounds, N-halamines, nanoparticles of noble metals, and metal oxides, among others. These products however, can suffer in the marketplace due to limited efficacy, microbial resistance mechanisms and cost of manufacture among other issues.

As a method of combating this growth in micro-organisms in textile such as surgical face masks, reusable face masks, hospital gowns, surgical aprons, surgical drapes, and curtains, the present disclosure seeks to incorporate one or more agents capable of providing antimicrobial and/or self-cleaning benefits in a robust, consistent, and efficacious manner One or more of these agents utilize visible light-activated finishes as antimicrobial, antifungal, and/or antiviral agents. The present disclosure further includes various ways to attach the antimicrobial finishes to textiles of different composition in a more robust manner that also results in a more consistent delivery of antimicrobial efficacy.

Thus, in view of the problems and disadvantages associated with prior art devices, the present invention was conceived and one of its objectives is to provide a textile substrate combined, finished, coated, or otherwise impregnated with a predetermined volume and/or concentration of formula to bestow upon the substrate certain antimicrobial properties.

It is still another objective of the present invention to provide an antimicrobial substrate comprising a non-woven, knitted, woven, or foam material processible under common manufacturing conditions. The antimicrobial substrate may be formed from polyester, polyethylene, polypropylene, rayon, acrylic, nylon, cotton, regenerated cellulose, wood pulp, and blends thereof.

It is yet another objective of the present invention to provide an antimicrobial substrate including a binder appropriate for use as a finish, coating and/or impregnating emulsion. The binder may be defined as acrylic, polyurethane, polyvinyl chloride, vinyl acetate, vinyl-acetate ethylene (VAE), and combinations thereof.

It is a further objective of the present invention to provide an antimicrobial substrate further including a binder and auxiliary chemistry such as ethylenediaminetetraacetic acid (EDTA) and/or salts thereof to provide enhanced antibacterial effects against gram negative bacteria. These coatings may be applied to the substrate by methods such as dip, foam, knife, pad, or other acceptable methodologies as known in the art.

It is still a further objective of the present invention to provide an antimicrobial substrate in the nature of a facemask topically finished and/or coated with a reactive oxygen species-generating dye (ROS) such as the flourescein class of dyes including eosin, phloxine, calcein, fluorescein amidite, erythorisine and rose bengal, among others. Additionally, phenothiazine dyes may be used including methylene blue or porphyrin dyes including verteporfin. These dyes may be used alone or in combination with each other. These dyes are topically applied on the textile in a concentration between 0.01-5.0% on weight of substrate, and more preferably 0.05-2.5% on weight, of the finished substrate.

It is yet a further objective of the present invention to provide an antimicrobial substrate in the nature of a facemask topically coated with a reactive oxygen species-generating dye (ROS) such as rose Bengal, methylene Blue, or mixtures thereof are impregnated on the textile in a concentration between 0.01-5.0% on weight of substrate, and more preferably 0.05-2.5% on weight, of the finished substrate.

Various other objectives and advantages of the present invention will become apparent to those skilled in the art as a more detailed description is set forth below.

### SUMMARY OF THE INVENTION

The aforesaid and other objectives are realized by providing an antimicrobial substrate suitable for configuration as a portion of personal protective equipment (PPE), for example as an outer layer of a textile facemask, protective garment, or other textile. The antimicrobial substrate, preferably formed from polypropylene, polyethylene, and/or polyester, receives a topically applied finish containing a reactive oxygen species-generating dye (ROS) such as rose bengal in a finish coated on the textile in a concentration between 0.01-5.00% on weight of the finished substrate, to provide antimicrobial or self-cleaning benefits to the textile substrate. In one or more embodiments, the topical finish applied to the antimicrobial substrate further includes ethylenediaminetetraacetic acid (EDTA) and/or salts thereof as auxiliary chemistry to provide enhanced antibacterial effects against gram negative bacteria. In one or more embodiments, the topical finish applied to the antimicrobial substrate further includes a binder appropriate for use as a coating emulsion such as acrylic, polyurethane, polyvinyl chloride, vinyl acetate, vinyl-acetate ethylene (VAE), and potentially surfactants, detergents, dyes, pigments, preservatives, and combinations thereof.

In one or more alternative embodiments, differing methods may be desirable to affix the ROS dye to the antimicrobial substrate. For example, a ROS such as rose bengal may be reacted with 4-vinyl benzyl chloride to form a rose bengal vinyl benzyl dye. This dye may be polymerized with styrene to form a polystyrene co-polymeric mixture attractive for use as a dispersing dye. The mixture may be dissolved in an appropriate non-polar solvent such as dichloromethane (DCM), dichloroethane (DCE), toluene, and/or other non-polar solvents and applied to the textile, which could be formed from polyolefin, polypropylene, polyethylene, polyester, and other suitable synthetic fibers. Additionally, or in the alternative, a ROS such as rose bengal may be reacted with 4-vinyl benzyl chloride to form a rose bengal vinyl benzyl dye which in turn is then polymerized with 4-styrene sulfonic acid and a styrene-triazine monomer to form a functional substrate as a comonomer in a water-soluble polymer. The resultant water-soluble polymer may be used as a reactive substrate for cellulosic fibers. The water-soluble polymer is dissolved into an appropriate aqueous and/or alcohol solution and affixed to cellulosic textile formed from materials such as cotton, rayon, wood pulp, regenerated cellulosic fiber(s) and textile containing mixtures of said cellulosic fiber(s) and synthetic fiber(s).

### BRIEF DESCRIPTION OF THE DRAWINGS

FIG. 1 is a perspective view showing an exemplary embodiment of an antimicrobial substrate.

### DETAILED DESCRIPTION OF THE PREFERRED EMBODIMENT AND OPERATION OF THE INVENTION

Various exemplary embodiments of the present disclosure are described below. Use of the term “exemplary” means illustrative or by way of example only, and any reference herein to “the invention” is not intended to restrict or limit the invention to exact features or step of any one or more of the exemplary embodiments disclosed in the present specification. References to “exemplary embodiment”, “one embodiment”, “an embodiment”, “various embodiments”, and the like may indicate that the embodiment(s) of the invention so described may include a particular feature, structure, or characteristic, but not every embodiment necessarily incudes the particular feature, structure, or characteristic. Further, repeated use of the phrase “in one embodiment”, “in an exemplary embodiment”, or “in an alternative embodiment” do not necessarily refer to the same embodiment, although they may.

It is also noted that terms like “preferably”, “commonly”, and “typically” are not utilized herein to limit the scope of the invention or to imply that certain features are critical, essential, or even important to the structure or function of the invention. Rather, these terms are merely intended to highlight alternative or additional features that may or may not be utilized in a particular embodiment of the present invention.

The present invention is described more fully hereinafter with reference to the accompanying figures, in which one or more exemplary embodiments of the invention are shown. Like numbers used herein refer to like elements throughout. The invention may, however, be embodied in many different forms and should not be construed as limited to the embodiments set forth herein; rather, these embodiments are provided so that this disclosure will be operative, enabling, and complete. Accordingly, the particular arrangements disclosed are meant to be illustrative only and not limited as to the scope of the invention, and any and all equivalents thereof. Moreover, many embodiments such as adaptations, variations, modifications, and equivalent arrangements will be implicitly disclosed by the embodiments described herein and fall within the scope of the instant invention.

Although specific terms are employed herein, they are used in a generic and descriptive sense only and not for the purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. Where only one item is intended, the terms “one and only one”, “single”, or similar language is used. When used herein to join a list of items, the term “or” denotes at least one of the items but does not exclude a plurality of items of the list.

For exemplary methods or processes of the invention, the sequence and/or arrangement of steps described herein are illustrative and not restrictive. Accordingly, it should be understood that, although steps of various processes or methods may be shown and described as being in a sequence or temporal arrangement, the steps of any such processes or methods are not limited to being carried out in any particular sequence or arrangement, absent an indication otherwise. Indeed, the steps in such processes or methods generally may be carried out in various different sequences and arrangements while still falling within the scope of the present invention.

Additionally, any references to advantages, benefits, unexpected results, or operability of the present invention are not intended as an affirmation that the invention has previously been reduced to practice or that any testing has been performed. Likewise, unless stated otherwise, use of verbs in the past tense (present perfect or preterit) is not intended to indicate or imply that the invention has previously been reduced to practice or that any testing has been performed.

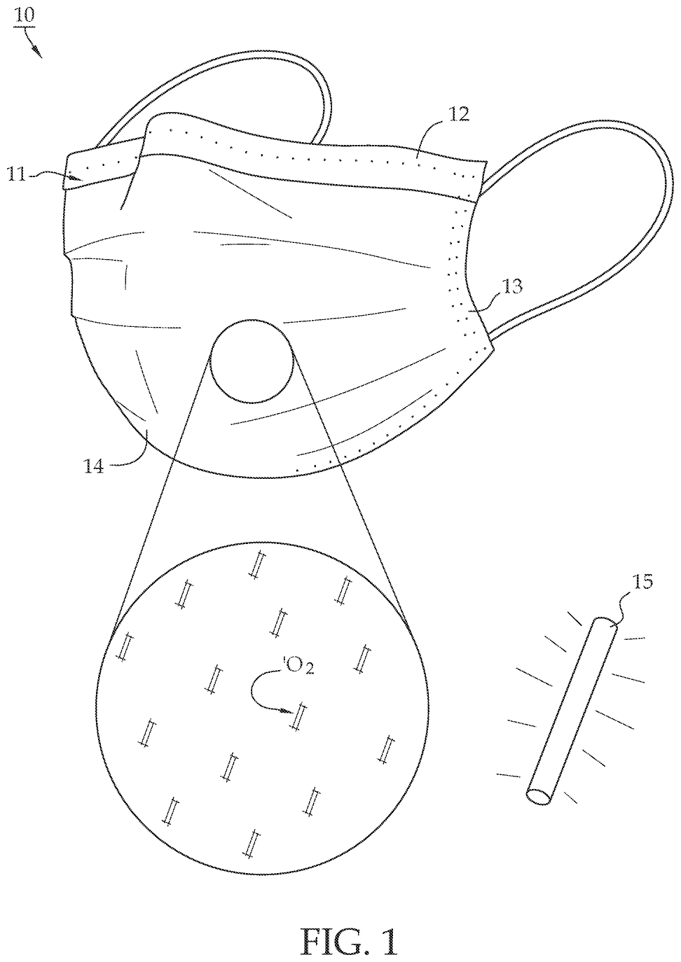

For a better understanding of the invention and its operation, the preferred embodiment of antimicrobial substrate 10 shown in FIG. 1 is in the nature of a portion 11 of personal protective equipment (PPE), and more preferably in the nature of an exterior or interior portion of a facemask as known in the art. Embodiments of antimicrobial substrate 10 may be formed from polyester, polyethylene, polypropylene, rayon, acrylic, nylon, cotton, wood pulp, regenerated cellulose, and blends thereof. However, other embodiments of other natural and synthetic textile(s) and cellulosic portion(s) are similarly included within the scope of the instant disclosure. Embodiments of antimicrobial substrate 10 are preferably treated with a coating, finish, or formula in sufficient volume and/or concentration to bestow upon antimicrobial substrate 10 certain antimicrobial properties.

In certain preferred embodiments, it may be advantageous to utilize a binder 12 to facilitate the attachment or connection of a reactive oxygen species-generating dye (ROS) 13 such as (but not limited to) a flourescein class of dyes including eosin, phloxine, calcein, fluorescein amidite, erythorisine and rose bengal, among others. Additionally, phenothiazine dyes may be used including methylene blue or porphyrin dyes including verteporfin. These dyes may be used alone or in combination with each other. In one embodiment, either rose bengal or methylene blue are attached to, or impregnated in a portion 11 of antimicrobial substrate 10. Acceptable species of binder 12 include, but are not limited to, one or more binders defined as acrylic, polyurethane, polyvinyl chloride, vinyl acetate, vinyl-acetate ethylene (VAE), and combinations thereof. Additionally, or in the alternative, certain auxiliary chemistry 14 such as ethylenediaminetetraacetic acid (EDTA) and/or salts thereof, commercial surfactants, pigments, and detergents, may be added into the topically applied finish including binder 12 to provide enhanced antibacterial effects, for example against gram negative bacteria. EDTA may be added to the finish coated on the textile in a concentration between 0.1-10.0% weight of the finished substrate. Regardless as to whether the resulting finish, formulation, or coating does or does not include a binder, the finish is preferably dispersed in sufficient volume and/or concentration to bestow upon the substrate 10 certain antimicrobial properties. In one embodiment, the topically applied finish described above is preferably defined as a visible light-activated finish containing rose bengal in a concentration of 1.0-30.0% on weight of the finished substrate, and more preferably 1.0-10.0% on weight of the finished substrate, and more preferably still 0.01-5.0% on weight of the finished substrate, and most preferably of all 0.05-2.5% on the weight of the finished substrate, to provide antimicrobial or self-cleaning benefits to the textile substrate 10. In a different embodiment, the finish formulation described above is preferably defined as a visible light-activated coating containing methylene blue in a concentration of 1.0-30.0% on weight of the finished substrate, and more preferably 1.0-10.0% on weight of the finished substrate, and more preferably still 0.01-5.0% on weight of the finished substrate, and most preferably of all 0.05-2.5% on the weight of the finished substrate, to provide antimicrobial or self-cleaning benefits to the textile substrate 10. In any of these formulations, binder 12 may be added to the formulation in a concentration between 1.0-30.0% weight solid, and more preferably 0.01-5.0% on weight solid. Additionally, the inert matrix in which the application materials are mixed in the formulation as described above may be comprised of solvents including (but not limited to) water, methanol, ethanol, propanol, butanol, toluene, dichloromethane, or mixtures thereof. Formulation(s) may contain pH adjustors (acids and alkalis), processing aids (salts and minerals), polymers (e.g., polyvinyl alcohols), preservatives, pigments, and/or surfactants. In any of the instantly disclosed formulations, a preservative may be added in a concentration between 0.1-1.0%, and more preferably of 0.01-1.0%. Regardless as to the specific amounts of one or more of the aforementioned components of the finish as described above, it is therefore desirable for the final finish to be physiologically relevant to one or more microbes as disclosed herein. The term “physiologically relevant” as used in this context is construed to mean capable of reducing the one or more microbes that experience the topically applied finish by at least a factor of two (2) log compared a placebo group of the one or more microbes that do not experience one or more embodiments of the finish as described herein.

Differing application methods of connecting, attaching, or applying the topically applied finish are considered within the scope of the instant disclosure. Embodiments of the formulation as described above may be distributed by techniques including (but not limited) pad application, coating, foam, dip, or knife application(s). In one or more alternative embodiments, a ROS such as rose bengal may be reacted with 4-vinyl benzyl chloride to form a rose bengal vinyl benzyl dye which in turn is then polymerized with 4-styrene sulfonic acid and a styrene-triazine monomer to form a functional substrate as a comonomer in a water-soluble polymer. The water-soluble polymer may be used as a reactive substrate for cellulosic fibers. The water-soluble polymer is dissolved into an appropriate aqueous and/or alcohol solution and affixed to cellulosic textile formed from materials such as cotton, rayon, regenerated cellulosic fiber(s), and textile containing mixtures of said cellulosic fiber(s) and synthetic fiber(s). A schematic representation of the aforementioned reaction is included below as Example 1.

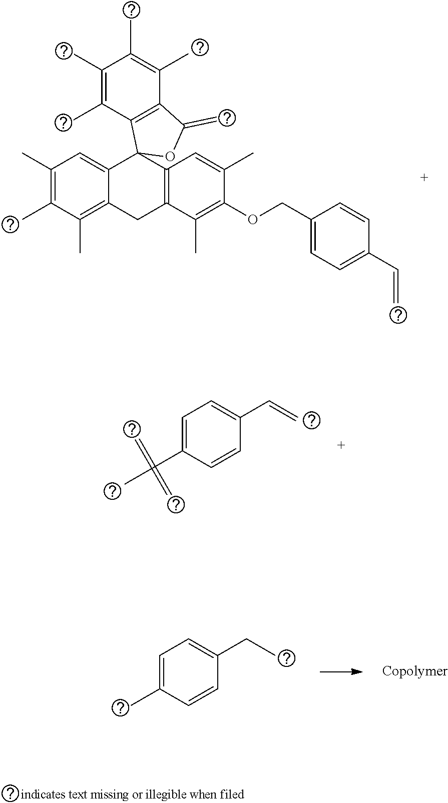

Triazine Monomer Ideas

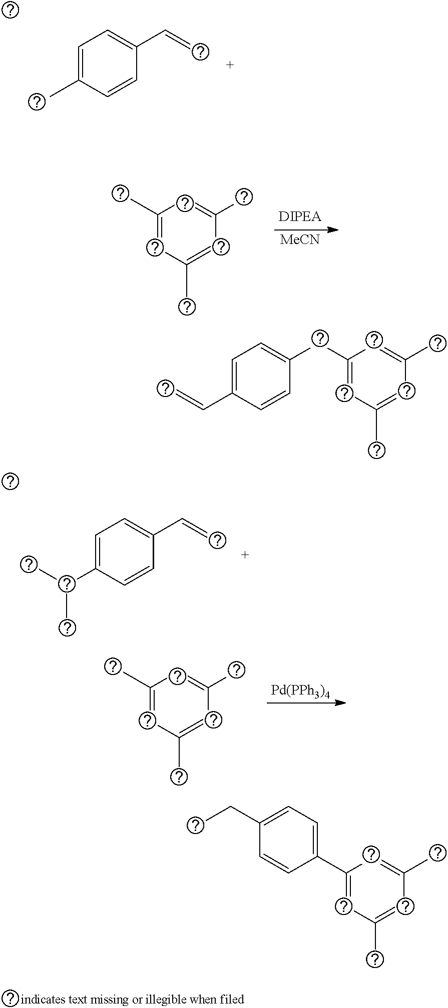

### EXAMPLE 1

Additionally, or in the alternative, a ROS such as rose bengal may be reacted with 4-vinyl benzyl chloride to form a rose bengal vinyl benzyl dye. This dye may be polymerized with styrene to form a polystyrene co-polymeric mixture attractive for use as a dispersing dye. The mixture may be dissolved in an appropriate non-polar solvent such as dichloromethane (DCM), dichloroethane (DCE), toluene, and/or other non-polar solvents and applied to portion 11, which may be formed from polyolefin, polypropylene, polyethylene, polyester, and/or other suitable synthetic fibers. A schematic representation of the aforementioned reaction is included below as Example 2.

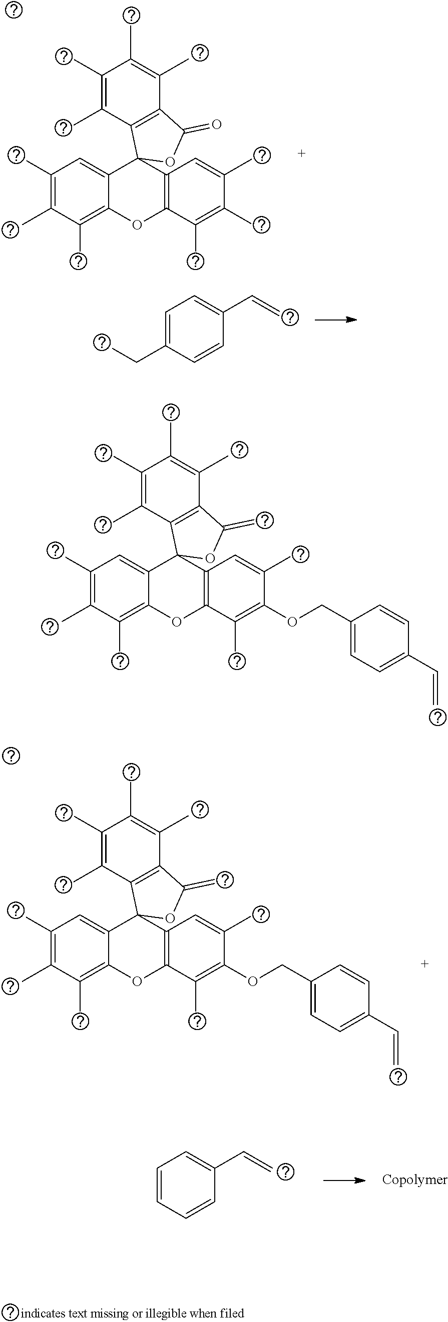

### EXAMPLE 2

In use, one or more portions 11 of antimicrobial substrate 10, preferably formed from polypropylene, polyethylene, and/or polyester, is coated with a topically applied finish including a dye or resin activated by electromagnetic radiation in the visible light region of the light spectrum containing a reactive oxygen species-generating dye (ROS) such as rose bengal in a concentration between 0.01-5.0% on weight of substrate, and more preferably 0.05-2.5% on weight, of the finished substrate, to provide antimicrobial or self-cleaning benefits to the textile substrate. The finish applied to the antimicrobial substrate 10 further includes Ethylenediaminetetraacetic acid (EDTA) and/or salts thereof as auxiliary chemistry 13 to provide enhanced antibacterial effects against gram negative bacteria. In one or more embodiments, the finish applied to the antimicrobial substrate further includes a binder 12 appropriate for use as a coating emulsion such as acrylic, polyurethane, polyvinyl chloride, vinyl acetate, vinyl-acetate ethylene (VAE), and combinations thereof, and in one or more other embodiments other acceptable surfactants and/or detergents. After use, the antimicrobial substrate 10 is exposed to a light stimulus 15 sufficient to generate the emission of singlet oxygen species (¹O₂) that generate substantial and robust antimicrobial activity against a broad variety of micro-organisms, including bacteria, viruses, fungi, providing meaningful self-cleaning benefits over the intended use of said textile. In one embodiment, the activation stimulus is defined as electromagnetic spectrum activation that occurs in the visible light portion of the light spectrum, defined between 450 and 700 nanometers (nm). In one preferred embodiment, the activation stimulus is defined as electromagnetic spectrum activation that occurs in the visible light portion of the light spectrum, defined between 490-575 nm. In an alternate embodiment, the activation stimulus is defined as electromagnetic spectrum activation that occurs in the visible light portion of the light spectrum, defined between 550-700 nm. The repeated exposure of antimicrobial substrate 10 to the preferred electromagnetic activation, and the continual emission of singlet oxygen as described above, provides the basis for the determination that the instant disclosure may be considered a “self-cleaning” antimicrobial substrate, and is considered a great advantage over the prior art.

For the purposes of describing and defining the present invention it is noted that the use of relative terms, such as “substantially”, “generally”, “approximately” and the like, are utilized herein to represent an inherent degree of variability that may be attributed to any quantitative comparison, value, measurement, or other representation. These terms are also utilized herein to represent the degree by which a quantitative representation may vary from a stated reference without resulting in a change in the basic function of the subject matter at issue.

Exemplary embodiments of the present invention are described above and below. No element, act, or instruction used in this description should be construed as singularly important, necessary, critical, or essential to the invention as a whole unless explicitly described as such. Although only a few of the exemplary embodiments have been described in detail herein, those skilled in the art will readily appreciate that many modifications are possible in these exemplary embodiments without materially departing from the novel teachings and advantages of this invention. The illustrations and examples provided herein are for explanatory purposes and are not intended to limit the scope of any subsequent claim. Accordingly, all such modifications are intended to be included within the scope of this invention.

Examples 3-13 with corresponding Tables 1-11 appear on the following pages.

### EXAMPLE 3

A polypropylene nonwoven textile 32 grams per square meter (GSM) was coated with a topically applied finish in a concentration of 7.42% on weight of the finished substrate with the dye defining 0.55% on weight of the finished substrate to form an antimicrobial substrate.

The resultant substrates are cut to 5×5 cm square size and placed in a 100 mm sterile petri dish before being inoculated with a known titer of Staphylococcus aureus. The inoculated substrates are exposed to electromagnetic radiation in the visible region under ambient conditions. The light source utilized is a 4100K fluorescent light at 32 Lumen output. At the end of the specified exposure time, the substrate is extracted with media, and the extraction plated and incubated. Microbial loading after treatment is determined and a log reduction is noted. Results are listed in Table 1. Increasing the contact time of the inoculum with the antimicrobial substrate from 15 minutes to 30 minutes or 60 minutes increases the efficacy of the substrate against Staphylococcus aureus. Likewise, removing the electromagnetic stimuli drastically reduces the antimicrobial efficacy of the substrate even with a long contact time.

TABLE 1

| | | | | | Light |
|---|---|---|---|---|---|
| | Contact | Initial | Final | Log | Intensity |
| Sample | time (min) | loading (log) | loading (log) | Reduction | (Lumens) |
| 1 | 15 | 7.59 | 7.32 | 0.27 | 32 |
| 2 | 30 | 6.81 | 5.40 | 1.41 | 32 |
| 3 | 60 | 7.83 | <5.3 | >2.53 | 32 |
| 4 | 60 | 7.83 | 7.53 | 0.21 | 0 |

### EXAMPLE 4

A polypropylene nonwoven textile 32 grams per square meter (GSM) was coated with a topically applied finish in a concentration of 7.42% on weight of the finished substrate with the dye defining varying % on weight of the finished substrate to form an antimicrobial substrate.

The resultant substrates are cut to 5×5 cm square size and placed in a 100 mm sterile petri dish before being inoculated with a known titer of Staphylococcus aureus. The inoculated substrates are exposed to electromagnetic radiation in the visible region under ambient conditions for 30 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Results are listed in Table 2. Increasing the weight % of rose bengal impregnated on the antimicrobial substrate increases the efficacy of the substrate against Staphylococcus aureus.

TABLE 2

| | Substrate % | Initial | Final | |
|---|---|---|---|---|
| | On Weight | loading | loading | |
| Sample | rose bengal | (log) | (log) | Log Reduction |
| 5 | 0.11 | 4.44 | 2.43 | 2.01 |
| 6 | 0.27 | 4.44 | 1.30 | 3.14 |
| 7 | 0.55 | 4.44 | 1 | 3.44 |
| 8 | 0.82 | 4.44 | <0.70 | >3.74 |

### EXAMPLE 5

A polypropylene nonwoven textile 32 grams per square meter (GSM) was coated with a topically applied finish in a concentration of 7.42% on weight of the finished substrate with the dye defining 0.55% on weight of the finished substrate to form an antimicrobial substrate.

The resultant substrates are cut to 5×5 cm square size and placed in a 100 mm sterile petri dish before being inoculated with a known titer of Pseudomonas aeruginosa. The inoculated substrates are exposed to electromagnetic radiation in the visible region under ambient conditions for 30 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Results are listed in Table 3. Incorporation of EDTA in either of the tested weight % in the desired solutions and impregnated on the antimicrobial substrate greatly increases the efficacy of the substrate against Pseudomonas aeruginosa.

TABLE 3

| | Substrate | Initial | Final | |
|---|---|---|---|---|
| | Weight % | loading | loading | Log |
| Sample | EDTA | (log) | (log) | Reduction |
| 9 | 0 | 5.85 | 5.72 | 0.13 |
| 10 | 0.59 | 5.50 | 0.70 | 4.80 |
| 11 | 1.09 | 5.50 | <0.70 | >4.80 |

### EXAMPLE 6

A polypropylene nonwoven textile 24 grams per square meter (GSM) was coated with a topically applied finish in a concentration of 9.89% on weight of the finished substrate with the dye defining 0.64% on weight of the finished substrate to form an antimicrobial substrate.

The resultant substrates are cut to 5×5 cm square size and placed in a 100 mm sterile petri dish before being inoculated with a known titer of Staphylococcus aureus. The inoculated substrates are exposed to electromagnetic radiation in the visible region under ambient conditions for 60 minutes. Varying light sources and color temperatures are explored with intensity kept constant at 32 Lumens. At the end of the specified exposure time, the substrates are extracted with media, and the extraction plated and incubated. Microbial loading after treatment is determined and a log reduction is noted. Results are listed in Table 4. Variation in the light source or correlated color temperature did not have a noticeable impact on the efficacy of the substrate against Staphylococcus aureus.

TABLE 4

| | | | Initial | Final | | | |
|---|---|---|---|---|---|---|---|
| | | Color | loading | loading | | Log | |
| | Light | Temperature | Average | Average | | Reduction | |
| Sample | Source | (K) | (log) | (log) | Replicates | Average | P Value |
| 12 | Fluorescent | 4100 | 4.87 | 1.69 | 3 | 3.18 | 1.93E−08 |
| 12 | LED | 4000 | 5.06 | 1.92 | 2 | 3.13 | 4.19E−05 |
| 12 | Fluorescent | 6500 | 5.08 | 1.57 | 2 | 3.51 | 6.57E−06 |

### EXAMPLE 7

A polypropylene nonwoven textile 17 grams per square meter (GSM) was coated with a topically applied finish in a concentration of 13.97% on weight of the finished substrate with the dye defining 0.78% on weight of the finished substrate to form an antimicrobial substrate.

The resultant substrates are cut to 5×5 cm square size and placed in a 100 mm sterile petri dish before being inoculated with a known titer of various microbes. The inoculated substrates are exposed to electromagnetic radiation in the visible region under ambient conditions for 30 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Results are listed in Table 5. Antimicrobial activity against various organisms was still observed with addition of pigment to the substrate.

TABLE 5

| | Substrate | | Initial | Final | | | |
|---|---|---|---|---|---|---|---|
| | % on | | loading | loading | | Log | |
| | Weight | | Average | Average | | Reduction | |
| Sample | Pigment | Organism | (log) | (log) | Replicates | Average | P Value |
| 13 | 0.02 | Staphylococcus aureus | 4.89 | 2.81 | 1 | 2.08 | |
| 14 | 0.03 | Staphylococcus aureus | 4.71 | 2.68 | 6 | 2.03 | 6.92E−03 |
| 14 | 0.03 | Pseudomonas aeruginosa | 5.35 | 1.53 | 4 | 3.82 | 6.92E−04 |
| 14 | 0.03 | Candida albicans | 3.58 | <1.0 | 3 | >2.58 | Identical data |
| | | | | | | | all replicates |
| 14 | 0.03 | Human Coronavirus, | 4.63 | <1.00 | 2 | >3.63 | 8.77E−02 |
| | | Strain 229E | | | | | |

### EXAMPLE 8

A polypropylene nonwoven textile 22 grams per square meter (GSM) was coated with a topically applied finish in a concentration of 10.79% on weight of the finished substrate with the dye defining 0.66% on weight of the finished substrate to form an antimicrobial substrate.

In another embodiment, a two-coat system is employed. A polypropylene nonwoven textile (22 GSM) was procured. This was coated with a topically applied finish in a concentration of 7.7% on weight of the finished substrate with the dye defining 0.5% on weight of the single coated substrate to form an antimicrobial substrate. The single coated substrate is coated with a topically applied finish to form a two-coat system in a concentration of 59.0% on weight of the finished substrate with the dye defining 0.17% on weight of the finished substrate.

The resultant substrates are cut to 5×5 cm square size and placed in a 100 mm sterile petri dish before being inoculated with a known titer of microbe. The inoculated substrates are exposed to electromagnetic radiation in the visible region under ambient conditions for 60 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Additionally, dry crocking of the resultant fabric is determined in accordance with AATCC Method 8: Colorfastness to Crocking: AATCC Crockmeter Method. Grades are assigned based on a sliding scale from 1-5; 1 recorded as low colorfastness and 5 corresponding to excellent colorfastness. Results are listed in Table 6. Robust, repeatable antimicrobial activity against all studied organisms was observed utilizing both the one and two-coat systems. Dry crocking was noticeably improved when a two-coat system was employed.

TABLE 6

| | | | Initial | Final | | | | |
|---|---|---|---|---|---|---|---|---|
| | | | loading | loading | Number | Log | | Dry |
| | | | Average | Average | of | Reduction | | Crocking |
| Sample | Coats | Organism | (log) | (log) | Replicates | Average | P Value | (1-5) |
| 15 | 1 | Staphylococcus aureus | 4.87 | 1.69 | 3 | 3.18 | 1.93E−08 | 2 |
| 15 | 1 | Pseudomonas aeruginosa | 5.39 | 2.06 | 3 | 3.33 | 2.52E−09 | 2 |
| 16 | 2 | Staphylococcus aureus | 4.88 | 1.79 | 3 | 3.09 | 4.66E−04 | 4 |
| 16 | 2 | Pseudomonas aeruginosa | 5.42 | 1.85 | 3 | 3.58 | 4.33E−03 | 4 |

### EXAMPLE 9

A polypropylene nonwoven textile 26 grams per square meter (GSM) was coated with a topically applied finish in a concentration of 9.13% on weight of the finished substrate with the dye defining 0.57% on weight of the finished substrate to form an antimicrobial substrate.

The resultant substrates are cut to 5×5 cm square size and placed in a 100 mm sterile petri dish before being inoculated with a known titer of microbe. The inoculated substrates are exposed to electromagnetic radiation in the visible region under ambient conditions for 60 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Results are listed in Table 7. Robust, repeatable antimicrobial activity against all studied organisms was observed.

TABLE 7

| | | Initial | Final | | | |
|---|---|---|---|---|---|---|
| | | loading | loading | Number | Log | |
| | | Average | Average | of | Reduction | |
| Sample | Organism | (log) | (log) | Replicates | Average | P Value |
| 17 | Staphylococcus aureus | 4.78 | 1.20 | 9 | 3.58 | 2.48E−05 |
| 17 | Pseudomonas aeruginosa | 5.45 | 1.03 | 12 | 4.42 | 2.08E−13 |
| 17 | Candida albicans | 3.49 | 1.16 | 13 | 2.33 | 1.55E−16 |

### EXAMPLE 10

A polypropylene nonwoven textile 26 grams per square meter (GSM) was coated with a topically applied finish in a concentration of 9.13% on weight of the finished substrate with the dye defining 0.57% on weight of the finished substrate to form an antimicrobial substrate.

The resultant substrates are cut to 5×5 cm square size and placed in a 60 degrees Celsius (° C.) incubator for a specified length of time. After the allotted time, the samples are removed and placed in a 100 mm sterile petri dish before being inoculated with a known titer of microbe. The inoculated substrates are exposed to electromagnetic radiation in the visible region under ambient conditions for 60 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Results are listed in Table 8. Robust, repeatable antimicrobial activity was observed after aging under accelerated conditions for both one and two months against Staphylococcus aureus and Pseudomonas aeruginosa.

TABLE 8

| | | | Initial | Final | | | |
|---|---|---|---|---|---|---|---|
| | Aging at | | loading | loading | Number | Log | |
| | 60 C. | | Average | Average | of | Reduction | |
| Sample | (month) | Organism | (log) | (log) | Replicates | Average | P Value |
| 17 | 1 | Staphylococcus aureus | 4.88 | 1.48 | 3 | 3.41 | 4.51E−09 |
| 17 | 2 | Staphylococcus aureus | 5.64 | 1.05 | 6 | 4.59 | 1.24E−05 |
| 17 | 2 | Pseudomonas aeruginosa | 5.46 | 1.00 | 6 | 4.46 | 1.44E−09 |

### EXAMPLE 11

A polypropylene nonwoven textile 17 grams per square meter (GSM) was coated with a topically applied finish in a concentration of 13.97% on weight of the finished substrate with the dye defining 0.83% on weight of the finished substrate to form an antimicrobial substrate.

The resultant substrates are cut to 5×5 cm square size and placed in a 100 mm sterile petri dish. The substrate is exposed to electromagnetic radiation in the visible region under ambient conditions for 9 hours. The light source utilized is a 4100K fluorescent light at 32 Lumen output. After 9 hours, the sample is inoculated with a known titer of Pseudomonas aeruginosa. The inoculated substrate is exposed to the same electromagnetic radiation source for an additional 30 minutes. At the end of the specified exposure time, the substrates are extracted with media, and the extraction plated and incubated. Microbial loading after treatment is determined and a log reduction is noted. Results are listed in Table 9. Robust antimicrobial activity was observed after extended electromagnetic radiation exposure for 9 hours prior to inoculation.

TABLE 9

| | Electromagnetic | | | |
|---|---|---|---|---|
| | Radiation | Initial loading | Final loading | Log |
| | Exposure Prior to | Average | Average | Reduction |
| Sample | Inoculation (hours) | (log) | (log) | Average |
| 18 | 0 | 4.69 | <0.70 | >3.99 |
| 18 | 9 | 4.69 | <0.70 | >3.99 |

### EXAMPLE 12

A polypropylene nonwoven textile 26 grams per square meter (GSM) was coated with a topically applied finish in a concentration of 9.13% on weight of the finished substrate with the dye defining 0.57% on weight of the finished substrate to form an antimicrobial substrate.

The resultant substrate is used as the outer layer construction of a three-layer surgical mask. The surgical mask construction consists of an inner face layer of polypropylene spunbonded nonwoven textile 30 grams per square meter (GSM), a filter layer consisting of melt blown nonwoven polypropylene textile 25 grams per square meter (GSM), and an outer layer consisting of the above mentioned treated antimicrobial substrate. The three layers are ultrasonically welded together and affixed with nose piece and ear loops to produce a functional three-layer surgical mask.

The resultant substrates are cut to 5×5 cm square size and placed in a 100 mm sterile petri dish before being inoculated with a known titer of microbe. The inoculated substrates are exposed to electromagnetic radiation in the visible region under ambient conditions for 60 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Results are listed in Table 10. Robust, repeatable antimicrobial activity against all studied organisms was observed.

TABLE 10

| | | Initial | Final | | | |
|---|---|---|---|---|---|---|
| | | loading | loading | Number | Log | |
| | | Average | Average | of | Reduction | |
| Sample | Organism | (log) | (log) | Replicates | Average | P Value |
| 19 | Staphylococcus aureus | 4.57 | 1.02 | 10 | 3.55 | 6.74E−05 |
| 19 | Pseudomonas aeruginosa | 5.43 | 1.18 | 10 | 4.25 | 2.03E−13 |
| 19 | Candida albicans | 5.18 | 2.04 | 3 | 3.15 | 8.15E−08 |

### EXAMPLE 13

A three-layer surgical mask was produced as exemplified in Example 12 (Sample 19). In addition, marketed masks with antimicrobial claims were procured.

The resultant substrates are cut to 5×5 cm square size and placed in a 100 mm sterile petri dish before being inoculated with a known titer of microbe. The inoculated substrates are exposed to electromagnetic radiation in the visible region under ambient conditions for 60 minutes. The light source utilized is a 4100K fluorescent light at 32 Lumen output. At the end of the specified exposure time, the substrates are extracted with media, and the extraction plated and incubated. Microbial loading after treatment is determined and a log reduction is noted. Results are listed in Table 11. Enhanced, repeatable antimicrobial activity of Sample 19 was observed in relation to activity observed with masks currently found in the marketplace.

TABLE 11

| | | Staphylococcus | Pseudomonas | Candida |
|---|---|---|---|---|
| | Actives | aureus | aeruginosa | albicans |
| Sample | Component | Log Reduction | Log Reduction | Log Reduction |
| 19 | rose bengal | 4.22 | 4.40 | 2.58 |
| | coating | | | |
| 20 | trivalent iodide | 0.85 | 0.91 | 1.26 |
| | coating | | | |
| 21 | silver chloride | 0.85 | 0.43 | 0.44 |
| | coating | | | |
| 22 | copper infused | −.04 | 0.33 | 0.17 |
| | fabric | | | |
| 23 | organosilane | 4.22 | 1.78 | 1.71 |
| | coating | | | |

1. An electromagnetic radiation-activated antimicrobial substrate comprising a textile substrate coated with a finish;
said finish comprised of a dye species configured to generate singlet oxygen when exposed to electromagnetic radiation; and
said substrate coated with the finish; wherein the antimicrobial substrate defines a predetermined amount of the finish that is physiologically relevant to one or more microbial species.

2. The antimicrobial substrate of claim 1, wherein the textile substrate is selected from the group consisting of polypropylene, polyethylene, polyester, rayon, acrylic, nylon, cotton, regenerated cellulose, wood pulp or a combination thereof.

3. The antimicrobial substrate of claim 1, wherein the finish further comprises a binder.

4. The antimicrobial substrate of claim 3, wherein the binder is selected from the group consisting of acrylic, polyurethane, polyvinyl chloride, vinyl acetate, vinyl-acetate ethylene, or combinations thereof.

5. The antimicrobial substrate of claim 4, wherein the finish is further comprised of one or more surfactants, detergents, dyes, preservatives, pigments, and combinations thereof.

6. The antimicrobial substrate of claim 1, wherein the finish further comprises ethylenediaminetetraacetic acid, ethylenediaminetetraacetic acid salts, or a combination thereof.

7. The antimicrobial substrate of claim 1, wherein the dye species is selected from the group consisting of rose bengal, eosin, phloxine, calcein, fluorescein amidite, erythorisine, methylene blue, verteporfin, other flourescein dyes, other phenothiazine dyes, other porphyrin dyes, or combination thereof.

8. The antimicrobial substrate of claim 7, wherein the dye species is defined as rose bengal, and the finish is defined as a topically applied finish that further comprises a water-soluble polymer that affixes the dye species to the textile substrate; wherein the rose bengal reacts with 4-vinyl benzyl chloride to form rose bengal vinyl benzyl dye; wherein the said rose bengal vinyl benzyl dye further reacts with a 4-styrene sulfonic acid and a styrene-triazine monomer to form a water-soluble polymer; and wherein the water soluble polymer facilitates affixing rose bengal to the textile substrate as a component of the topically applied finish.

9. The antimicrobial substrate of claim 8, wherein the water-soluble polymer is incorporated in the topically applied finish and affixed to the substrate; the substrate is selected from the group consisting of cotton, rayon, wood pulp, regenerated cellulosic fiber, synthetic fiber, or combination thereof.

10. The antimicrobial substrate of claim 7, wherein the dye species is defined as rose bengal, and the finish is defined as a topically applied finish that further compromises a polystyrene co-polymeric mixture that acts as a dispersing dye to affix to the textile substrate; and wherein the rose bengal reacts with 4-vinyl benzyl chloride to form rose Bengal vinyl benzyl dye; and wherein the rose bengal vinyl benzyl dye further reacts with styrene to form a polystyrene co-polymeric mixture attractive for use as a dispersing dye to affix to the textile substrate as a component of the topically applied finish.

11. The antimicrobial substrate of claim 10, wherein the polystyrene co-polymeric mixture is capable of being incorporated in the topically applied finish and affixed to the substrate; the substrate is selected from the group consisting of polypropylene, polyethylene, polyester, synthetic fiber, or combination thereof.

12. The antimicrobial substrate of claim 1, wherein the finish is coated between 0.01-5.00% on weight of the textile substrate.

13. The antimicrobial substrate of claim 1, wherein said finish is applied topically by dip, foam, knife and/or pad application methods.

14. The antimicrobial substrate of claim 1, wherein the finish further comprises a solvent.

15. The antimicrobial substrate of claim 14, wherein the solvent is selected from the group consisting of water, methanol, ethanol, propanol, butanol, toluene, dichloromethane, or mixtures thereof.

16. The antimicrobial substrate of claim 1, wherein the antimicrobial substrate is configured to cause at least a two log reduction in the number of fungal, viral, and bacterial entities in contact with the antimicrobial substrate when exposed to electromagnetic radiation for a specified exposure time.

17. The antimicrobial substrate of claim 16, wherein the antimicrobial substrate is configured to cause at least a two log reduction of Staphylococcus aureus, Pseudomonas aeruginosa, Candida albicans and/or Human Coronavirus, Strain 229E.

18. A method of forming an electromagnetic radiation-activated antimicrobial substrate comprising:
incorporating a dye species in a topically applied finish, said topically applied finish comprises an aqueous solution, alcohol solution, or combination thereof; and
coating the finish to a substrate;
wherein the antimicrobial substrate is coated with a predetermined amount of the finish that is physiologically relevant to one or more microbial species.

19. The method of claim 18 further comprising the step of adding a binder to the topically applied finish.

20. The method of claim 18 further comprising the step of adding ethylenediaminetetraacetic acid, ethylenediaminetetraacetic acid salts, or a combination thereof to the topically applied finish.

21. The method of claim 18 further comprising the steps of:
reacting the dye species with a 4-vinyl benzyl chloride to form a vinyl benzyl dye product;
reacting the vinyl benzyl dye product with a 4-styrene sulfonic acid and a styrene-triazine monomer to form a functional substrate as a comonomer in a water-soluble polymer; and
incorporating the water-soluble polymer in the topically applied finish.

22. The method of claim 21 further comprising affixing the topically applied finish to the substrate defined as a cellulosic textile formed from a group of materials consisting of cotton, rayon, regenerated cellulosic fiber(s) and textile containing mixtures of said cellulosic fiber(s) and synthetic fiber(s).

23. The method of claim 18 further comprising the steps of:
reacting the dye species with a 4-vinyl benzyl chloride to form a vinyl benzyl dye product;
reacting the vinyl benzyl dye product with a styrene to form a polystyrene co-polymeric mixture; and
incorporating the polystyrene co-polymeric mixture in the topically applied finish.

24. The method of claim 23 further comprising affixing the topically applied finish to the substrate defined as a polyolefin textile formed from a group of materials consisting of polypropylene, polyethylene, polyester, and textile containing mixtures of said synthetic fiber(s) and cellulosic fiber(s).
